(19) United States
(12) Patent Application Publication
(10) Pub. No.: US 2019/0321072 A1
(43) Pub. Date: Oct. 24, 2019

(54) APPARATUSES AND METHODS TO GUIDE PLACEMENT OF NEEDLES WITHIN THE BODY

(71) Applicant: Mayo Foundation for Medical Education and Research, Rochester, MN (US)

(72) Inventors: Kalpathi L. Venkatachalam, Jacksonville, FL (US); Vivek Gupta, Jacksonville, FL (US); Prasanna Vibhute, Jacksonville, FL (US); Alexander Bills, Atlanta, GA (US); Lucas Muller, Atlanta, GA (US); Dev Mandavia, Atlanta, GA (US); Cassidy Wang, Atlanta, GA (US); Marci Medford, Atlanta, GA (US)

(21) Appl. No.: 16/391,805

(22) Filed: Apr. 23, 2019

Related U.S. Application Data
(60) Provisional application No. 62/661,449, filed on Apr. 23, 2018.

(51) Int. Cl. A61B 17/34

(52) U.S. Cl. CPC A61B 17/3403; A61B 2017/00026; A61B 17/3401

(57) ABSTRACT

Apparatuses and methods can be used to guide placement of needles within the body. For example, this document describes apparatuses that measure impedance and/or force as a needle is passed through tissue to inform user of which anatomical space they are in. Low-cost portable bedside electro-physical apparatuses and methods for needle guidance and confirmation of successful placement in portions of the human body such as vessels and thecal sac are described herein.

### BACKGROUND

### 1. Technical Field

This document relates to apparatuses used to guide the placement of needles within the body. For example, this document relates to apparatuses that measure impedance and force as a needle is passed through tissue to inform user of which anatomical space they are in.

### 2. Background Information

Intravascular and intrathecal needle placement are among the commonest routine diagnostic and therapeutic medical procedures. Blind placement of intravascular and intrathecal needles can be quite challenging, especially in obese patients and deep locations. Currently, there is no simple bedside guidance method for needle placement in the arteries, veins or thecal sac.

The current technique of blind procedures involves positioning the patient, and palpating the spine to determine the region that feels the most accessible. After the physician must then guess the orientation and depth needed to insert the needle into the spine. As the needle is advanced, the physician must rely on tactile feedback to determine which anatomical areas of the spine the tip is in. This can lead to substantial guesswork, and trial and error by the physician constantly having to readjust the trajectory if not positioned correctly. In order to determine if the needle is in the epidural space, a technique called Loss of Resistance (LOR) is performed. This is done by the physician attaching a syringe filled with air or saline to the back of the needle prior to reaching the epidural space. The epidural space is then determined once a loss of resistance to flow is felt by the user indicating that the needle has passed from the ligamentum flavum into the epidural space. To determine if the subarachnoid space has been reached, a stylet is used to puncture the dura for presence of CSF fluid return. CSF return and LOR are the only measures of confirmation used to identify if the right location has been reached. While these techniques are sometimes effective, many times physicians cannot rely on these due to the potential of false losses and lack of CSF return. Additionally, the method of advancing the needle to the correct location is based upon tactile feedback, which can be inconsistent and dependent on patient.

The failure rate and difficulty of blind bedside intravascular and intrathecal access can be significantly reduced by guidance. These procedures are therefore increasingly being performed under radiological image guidance, which makes them expensive, limits availability, and exposes patients and personnel to radiation risk.

Neuraxial anesthesia is administered prior to childbirth to block pain and reduce patient stress. While the medication itself is effective, the techniques used to access the epidural and subarachnoid spaces are imprecise and often result in severe complications and substantial losses in time. These deficiencies are largely caused by the necessity to administer anesthesia blind—without fluoroscopic image guidance—to avoid exposing patients and babies to radiation.

### SUMMARY

This document describes apparatuses used to guide placement of needles within the body. For example, this document describes apparatuses that measure impedance and/or force as a needle is passed through tissue to inform user of which anatomical space they are in. Low-cost portable bedside electrophysical apparatuses and methods for needle guidance and confirmation of successful placement in areas of the body such as vessels and thecal sac are described herein. The apparatuses and methods are well-suited for widespread use in clinics, hospitals and research labs.

In one aspect, this disclosure is directed to an apparatus for identifying a position within a human body includes a handle; a needle extending from the handle, and circuitry within the handle. The needle can be releasably coupleable with the handle. The needle can include a first conductor and a second conductor. The first conductor can include a first electrically-uninsulated portion positioned along a shaft of the needle. The second conductor can include a second electrically-uninsulated portion positioned at a tip of the needle. The circuitry within the handle can be in electrical communication with the first and second conductors. The circuitry can be configured to determine a bioimpedance of tissue between the first and second electrically-uninsulated portions while the needle is inserted within the human body.

Such an apparatus for identifying a position within a human body may optionally include one or more of the following features. The handle can include one or more user feedback devices configured to provide an indication of the position within the human body based on the bioimpedance. The apparatus can also include a force sensor positioned to detect force exerted to the needle while the needle is being inserted within the human body. In some embodiments, the circuitry is configured to determine the detected force, and to determine the position within the human body based on both: (i) the bioimpedance of tissue between the first and second electrically-uninsulated portions while the needle is inserted within the human body, and (ii) the detected force.

In another aspect, this disclosure is directed to an apparatus for identifying a position within a human body. The apparatus includes: a handle; a needle extending from the handle; a force sensor positioned to detect force exerted to the needle while the needle is being inserted within the human body; and circuitry within the handle and in electrical communication with the force sensor; the circuitry configured to determine the detected force.

In another aspect, this disclosure is directed to a method of determining a position within a human body. The method comprises inserting the needle of any of the apparatuses described herein into the human body.

Particular embodiments of the subject matter described in this document can be implemented to realize one or more of the following advantages. The devices and techniques described herein provide an objective means for practitioners to identify entry into a target location (e.g., for administration of neuraxial anesthesia). As a result, the devices and techniques can improve clinician confidence, minimize complication rates, and eliminate the steep learning curve of current practice.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of an example device for guiding the placement of needles within a body in accordance with some embodiments provided herein.

FIG. 2 is a side view of an example bioimpedance-based device for guiding the placement of needles within a body in accordance with some embodiments provided herein.

FIG. 3 is a perspective view of the device of FIG. 2.

FIG. 4 provides enlarged views of the needle of the device of FIG. 2.

FIG. 5 provides another enlarged view of the needle of the device of FIG. 2.

FIG. 6 provides a perspective view of the inside of the handle of the device of FIG. 2.

FIG. 7 shows an example force-based device for guiding the placement of needles within a body in accordance with some embodiments provided herein.

Like reference numbers represent corresponding parts throughout.

### DETAILED DESCRIPTION

This document describes apparatuses used to guide the placement of needles within the body. In some embodiments, the apparatuses can differentiate between tissue layers using force and/or impedance measurements, among many other potential characterization techniques. For example, this document describes apparatuses that measure impedance and/or force as a needle is passed through tissue to inform user of which anatomical space they are in. In one example implementation, the apparatuses described herein are used to guide the placement of a needle for neuraxial anesthesia during labor and delivery.

Currently, blind placement of anesthesia is dependent on tactile feedback and the Loss of Resistance (LoR) encountered upon entry into the epidural space. Proficiency is gained and maintained only by continuous practice.

The devices described herein will allow physicians to identify entry into specific anatomical spaces by using bioelectric impedance analysis and/or force sensing (or other modalities). Bioelectric impedance analysis can be performed by generating alternating current (AC) in an electronics module (capital element) at the proximal end of the device with a frequency between 50 kHz and 100 kHz and limited to a maximum current of 100 μA. The epidural needle and an enclosed insulated stylet (disposable element) act as electrodes, which run current through the tissue at the tip of the needle. Tissue impedance will be measured in the electronics module. Based on results from clinical testing, tissue types can be characterized. An indicator system on the surface of the electronics module will notify the practitioner of current tissue location.

In some embodiments, the devices, systems, and methods described herein use a combination of force sensing and impedance to guide the user. Accordingly, the devices, systems, and methods described herein facilitate a way of navigating by stimulating a lower extremity and using a sensor array on the lower back to give the best location to start the access. In some embodiments, the devices described herein are configured to use any access needle in conjunction with a custom stylet attached to a syringe-like device that holds all of the electronics.

In some embodiments, force sensing is implemented by placing a force sensor such as a force-sensitive resistor (FSR) behind the needle to measure transmitted force. The signal will be processed in the electronics module and will be used to enhance the current method of identifying tissue layer.

The devices and techniques described herein provide an objective means for practitioners to identify entry into a target location (e.g., for administration of neuraxial anesthesia). As a result, the devices and techniques can improve clinician confidence, minimize complication rates, and eliminate the steep learning curve of current practice.

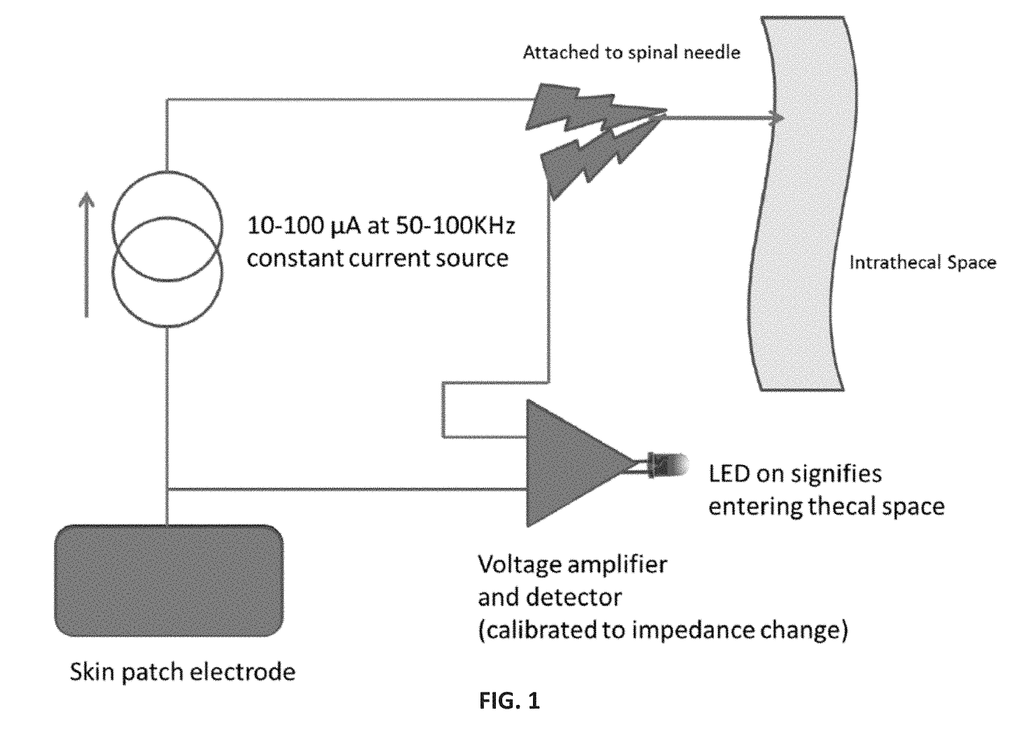

Referring to FIG. 1, in a first example embodiment (schematically represented here), a constant current of 10-100 μA at 50-100 KHz is injected between the tip of the access needle and a small remotely placed skin patch as the return electrode. The initial impedance between the needle and the skin patch will be high. As the needle approaches the thecal sac or large vascular structure, the impedance will steadily drop and abruptly fall upon penetration. This change in impedance can be measured by monitoring the voltage at the needle tip. Since a constant current is being injected, any drop in impedance will be reflected in a drop in voltage. This change can be measured and used to turn on an LED (light emitting diode) signaling that the intrathecal space has been reached. The risk to the patient is very minimal since currents at this frequency and amplitude are already being used in respiration monitors and intra-cardiac monitors to measure impedance. The whole device with battery and clips can be manufactured as a sterile disposable single-use system, or as a compact reusable system that can be sterilized.

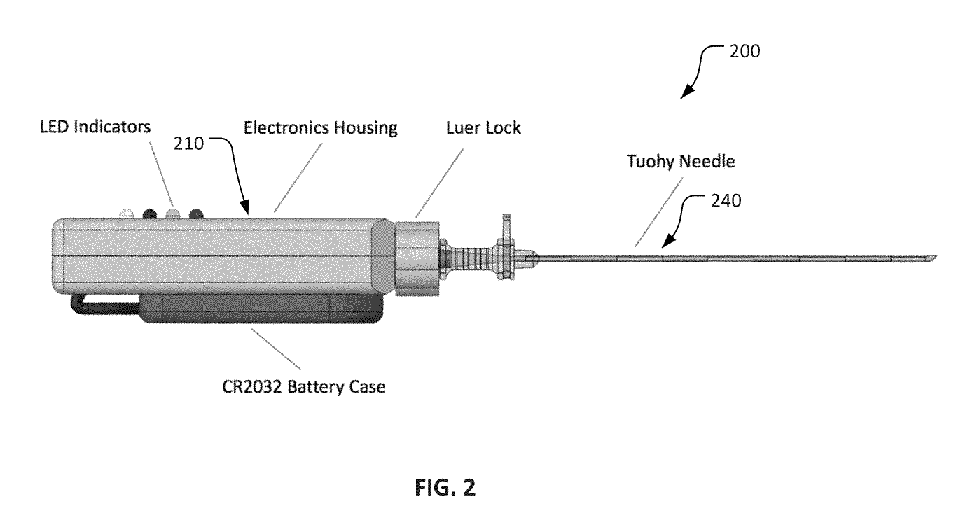

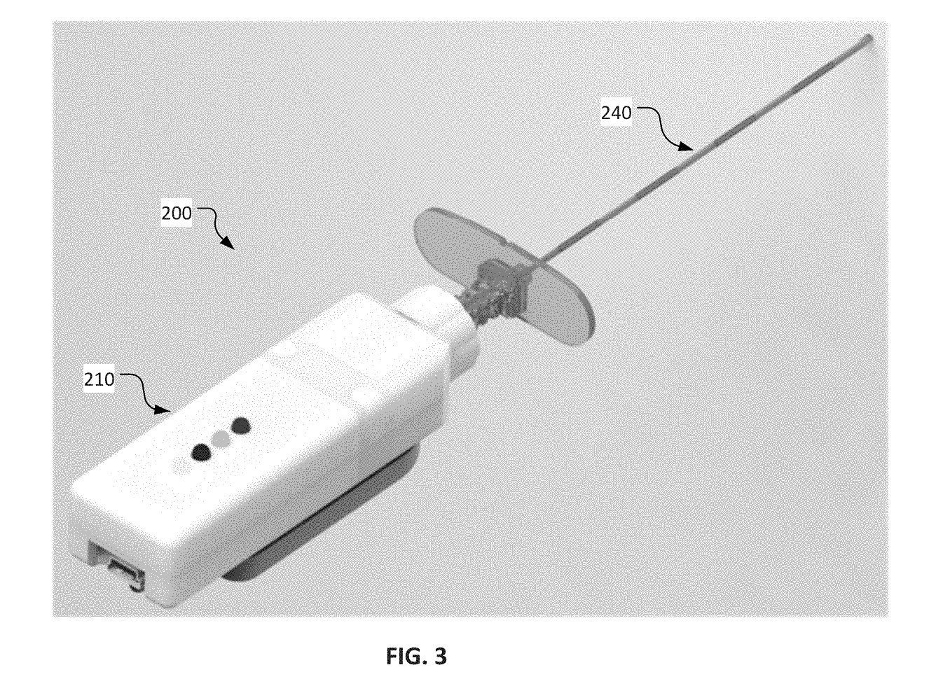

Referring to FIGS. 2 and 3, another example bioimpedance-based device 200 is depicted that can provide practitioners performing spinal access procedures (such as epidural anesthesia, spinal anesthesia, lumbar punctures, etc.) real-time information on which tissue layer the tip of the needle is currently in. Some embodiments of the device 200 provide guidance to reach, for example, the epidural/subarachnoid space using information that characterizes the different tissue layers. The device 200 will allow the practitioners to identify entry into specific anatomical spaces without the use of x-ray image-guidance.

The device 200 consists of two primary components, one reusable (the electronics housing module 210) and one disposable (the needle 240). The two components can be releasably coupled together using, for example, a luer lock connection as illustrated.

In some embodiments, the device 200 generates alternating current (AC) within a frequency 50-100 kHz and passes it through the substance at the tip of the needle 240 to characterize its impedance. As the needle 240 passes through tissues and anatomical structures of different compositions it can identify them based on their impedances. An indicator system on the handle 210 of the device 200 is used to signal to the user in what space the tip of the needle 240 currently resides.

The advantages of the illustrated device compared to current techniques include: provides tissue layer and anatomical space detection, allows the physician to hold the needle with both hands to increase stability and confidence, provides visual feedback for confirmation of placement within tissue layers that is not subjective, allows for easier training of medical personnel such as Residents, and eliminates physicians from having to rely on techniques such as tactile feedback and palpation which lack consistency.

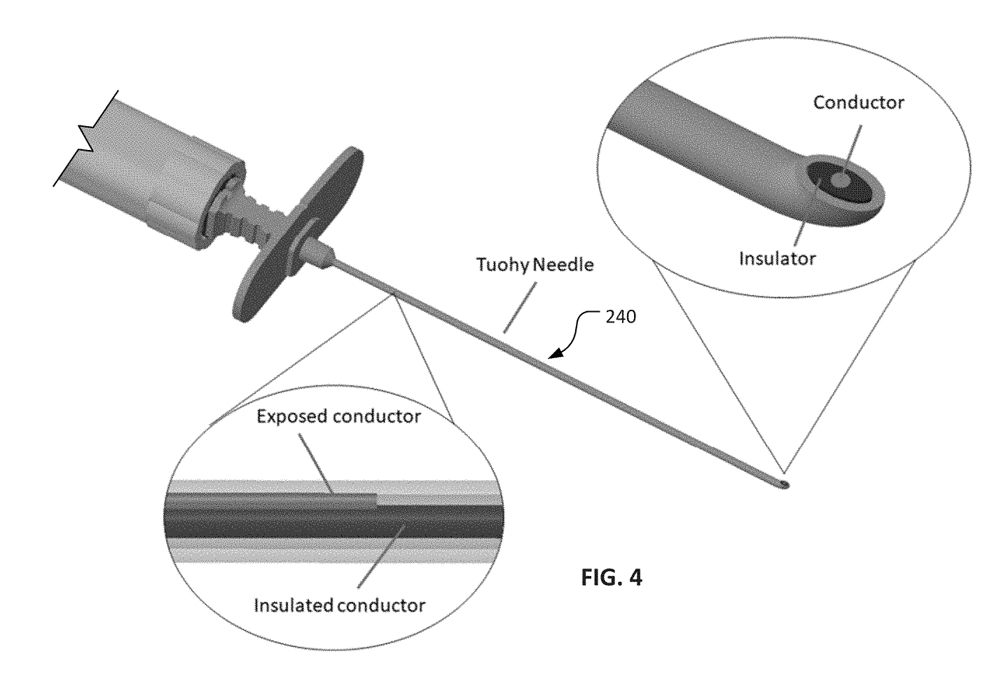

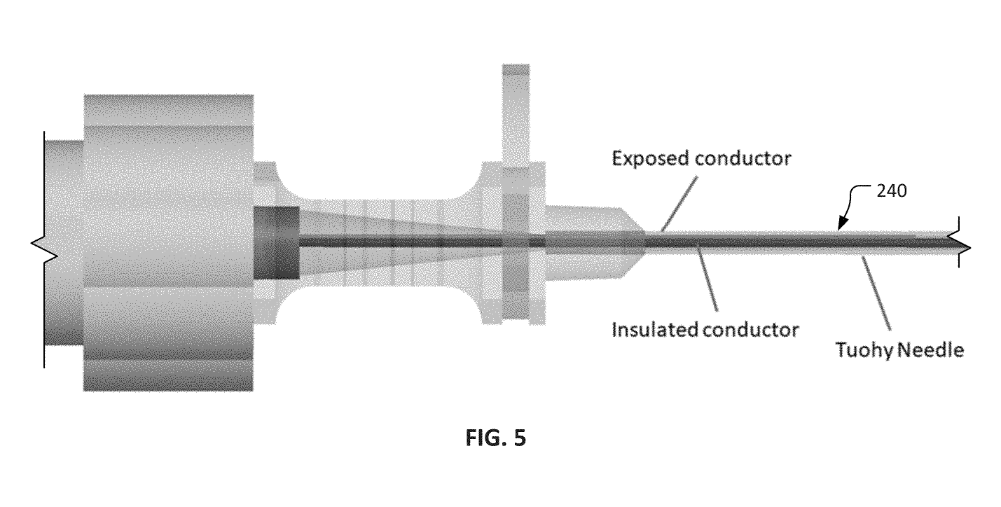

Referring also to FIGS. 4 and 5, the example device 200 measures impedance between two electrodes: a standard needle, and a custom stylet. The stylet is comprised of two conductive wires: (i) one exposed conductor in contact with the inside of the needle 240, and (ii) an insulated conductor. The insulated wire extends through the entire needle 240 and is cut flush with the opening at the needle tip, where the conductive component of the insulated wire is exposed.

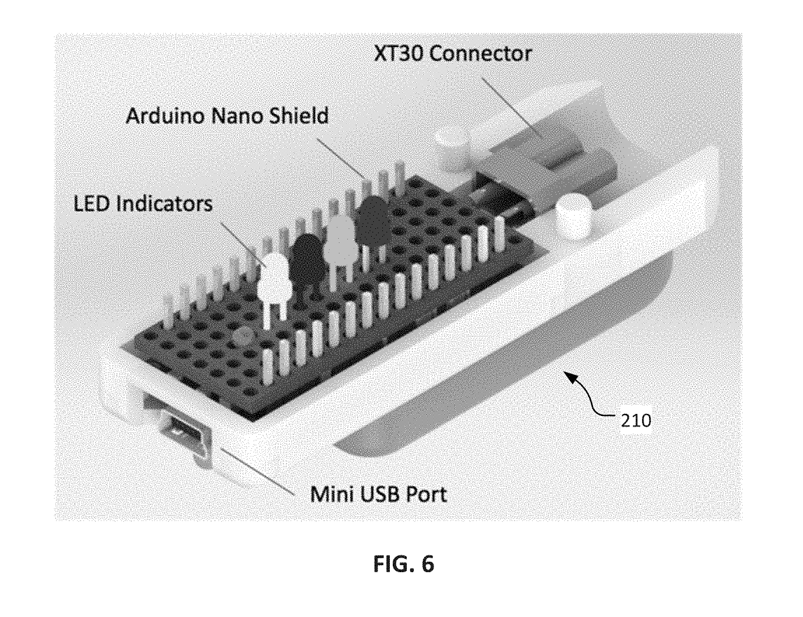

Referring also to FIG. 6, the handle 210 of the device 200 houses the electronics used to generate the AC current and measure impedance at the tip of the needle 240. A connector (e.g., a luer lock fitting as shown in FIG. 2) can be used to releasably couple the handle 210 and needle 240 such that the needle and stylet electrodes can be connected to the circuitry in the reusable handle 210.

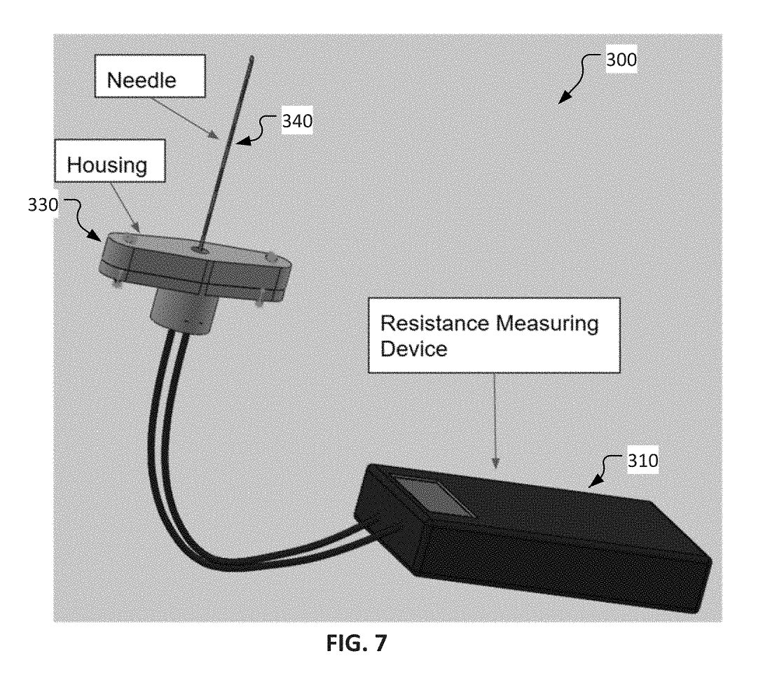

Referring to FIG. 7, another example device 300 can be used to identify entry into a target location (e.g., for administration of neuraxial anesthesia). The principle of operation of this system is to measure force transmitted through the needle 340 and to use the force magnitude and pattern to identify in which tissue layer the tip of the needle 340 is located and which layers it has passed through previously.

The device 300 uses a sensor to measure the force transmitted and passes the signal through an algorithm for processing. This method enhances the current method of identifying tissue layer location by providing quantitative information on where the tip of the needle 340 is located in the body.

In some embodiments, the force sensing of the device 300 can be accomplished using a force sensing resistor (FSR). The electrical resistance of the FSR changes with changing force applied to its surface. The resistance is read by some outside device (e.g. a microcontroller) and is processed to determine the tissues in which the needle 340 is located and through which it has passed through. To measure resistance, a resistor of known value is placed in series with the FSR and a known voltage is applied to the circuit. The outside device measures the voltage across the FSR, and determines the resistance.

The needle 340 is mounted to and extends from an outside housing 330, with the FSR located behind the needle in a position such that any force pushing on the needle from the tip in the direction of the handle will be opposed by a force between the FSR and the needle. Therefore, all the force transmitted through the needle will be passed through the FSR.

Other methods could also be used to calculate force passed through the needle 340. For example, strain gauges, piezoelectric sensors, elastic devices, magneto-elastic devices, and plastic deformation devices may also be used to measure forces transmitted through the needle 340. In addition, changes may also be implemented on the algorithm or the measurement device. For example, a wheatstone bridge, rather than a voltage divider, may be used to measure the resistance of and thereby the force on the FSR.

While the device 200 (FIGS. 2-6) is a bioimpedance-based device, and the device 300 (FIG. 7) is a force-based device, in some embodiments a single device can include the capacity to perform both bioimpedance-based measurements and force-based measurements. For example, in some embodiments the devices can measure impedance and force as a needle is passed through tissue to inform user of which anatomical space they are in.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. An apparatus for identifying a position within a human body, the apparatus comprising:
a handle;
a needle, releasably coupleable with the handle, the needle comprising a first conductor including a first electrically-uninsulated portion positioned along a shaft of the needle and a second conductor including a second electrically-uninsulated portion positioned at a tip of the needle; and
circuitry within the handle and in electrical communication with the first and second conductors, the circuitry configured to determine a bioimpedance of tissue between the first and second electrically-uninsulated portions while the needle is inserted within the human body.

2. The apparatus of claim 1, wherein the handle comprises one or more user feedback devices configured to provide an indication of the position within the human body based on the bioimpedance.

3. The apparatus of claim 1, further comprising a force sensor positioned to detect force exerted to the needle while the needle is being inserted within the human body.

4. The apparatus of claim 3, wherein the circuitry is configured to determine the detected force, and wherein the circuitry is configured to determine the position within the human body based on both: (i) the bioimpedance of tissue between the first and second electrically-uninsulated portions while the needle is inserted within the human body, and (ii) the detected force.

5. An apparatus for identifying a position within a human body, the apparatus comprising:
a handle;
a needle extending from the handle;
a force sensor positioned to detect force exerted to the needle while the needle is being inserted within the human body; and
circuitry within the handle and in electrical communication with the force sensor; the circuitry configured to determine the detected force.

6. A method of determining a position within a human body, the method comprising inserting the needle of the apparatus of any claim of claims 1 through 5 into the human body.
